(19) United States
(12) Patent Application Publication
(10) Pub. No.: US 2026/0097179 A1
(43) Pub. Date: Apr. 9, 2026

(54) Sleep Mask

(71) Applicant: SleepSanity LLC, Pompano Beach, FL (US)

(72) Inventors: Stephen A. Ziskind, Lexington, KY (US); Frederick Mackler, Boynton Beach, FL (US); Shuai Wang, Shenzhen City (CN)

(73) Assignee: SleepSanity LLC, Pompano Beach, FL (US)

(21) Appl. No.: 18/907,851

(22) Filed: Oct. 7, 2024

(51) Int. Cl. A61M 21/02; A61N 5/06

(52) U.S. Cl. CPC A61M 21/02; A61N 5/0613; A61M 2205/3303; A61M 2205/581; A61M 2205/587; A61M 2205/8237; A61N 2005/0626; A61N 2005/063; A61N 2005/0647; A61N 2005/0651; A61N 2005/0663

(57) ABSTRACT

A sleep mask is disclosed to improve the quality and effectiveness of sleep. The sleep mask includes a housing adapted to be worn by a user, at least one LED attached to the housing, and a power source attached to the housing and electrically connected to provide power to the at least one LED. The sleep mask further includes a silicone member attached to the housing. A lens with adjustable opacity may be arranged within a channel of the silicone member to transmit or block light. A fiber optic cable may also be arranged within a channel of the silicone member to transmit light from the at least one LED around at least a portion of the lens. A connector is arranged to connect the at least one LED to the fiber optic cable.

### BACKGROUND OF THE INVENTION

The present invention relates generally to the field of sleep masks to improve the quality and effectiveness of sleep.

According to a study conducted by the Centers for Disease Control and Prevention, approximately one-third of U.S. adults do not get enough sleep. In addition to cognitive impairment and mood change, individuals who sleep inadequately may have an increased risk of anxiety, depression, stress, heart disease, high blood pressure, diabetes, and other conditions. Thus, there is a substantial need for a device which facilitates restful sleep.

Conventional sleep masks may be made of cloth or another opaque material that is unable to vary light transmissivity over time. Thus, they require a user to put the mask on to darken the environment or to take the mask off to lighten it. Other existing sleep masks are unable to vary light intensity or color over time. Additionally, conventional sleep masks are unable to efficiently and uniformly transmit light to a user. Moreover, conventional sleep masks lack audio systems which are easily adjustable by a user to facilitate restful sleep or relaxation.

Accordingly, a need exists for a sleep mask which improves the quality and effectiveness of sleep. Particularly, a need exists for a sleep mask which has adjustable opacity and scientifically selected lighting that facilitate a user falling asleep and waking up. Additionally, a need exists for a sleep mask with an improved audio system that can be customized by a user. A need also exists for a sleep mask that provides user control of various features including, light transmissivity of a lens, light intensity, light color from LEDs, and/or audio output from an audio system. Moreover, a sleep mask system that includes an application for customizing such features is needed.

### BRIEF SUMMARY OF THE INVENTION

An embodiment in accordance with the present invention provides a sleep mask including a housing adapted to be worn by a user. The housing may be made of a material which is flexible and comfortable.

In one embodiment, the sleep mask may include a silicone member attached to the housing. The silicone member may have at least a first channel and a second channel therein. A lens may be arranged within the first channel and a fiber optic cable may be arranged within the second channel.

In one embodiment, at least a portion of the first channel may be substantially opaque and the second channel may be substantially translucent.

In another embodiment, the sleep mask may include at least one LED attached to the housing. The at least one LED may include a first LED and a second LED. In some embodiments, each of the first LED and the second LED may be configured to transmit light having a separate color. For example, the first LED may be configured to transmit an orange light, and the second LED may be configured to transmit a blue light.

The first LED may be configured to transmit light having a wavelength ranging from about 590 nm to 610 nm. The second LED may be configured to transmit light having a wavelength ranging from about 470 nm to 490 nm.

In some embodiments, the sleep mask may include a lens attached to the housing. The lens may have an adjustable opacity for selectively transmitting or blocking light waves. The lens may be arranged within the first channel of the silicone member. In a further embodiment, the lens may be made of polymer dispersed liquid crystal (PDLC).

In another embodiment, the sleep mask may include a fiber optic cable. In an embodiment, the fiber optic cable may be arranged within the second channel of the silicone member for transmitting light from the at least one LED around at least a portion of the lens. The fiber optic cable may be arranged to extend around at least 50% of the lens. In a further embodiment, the fiber optic cable may be arranged to extend around at least 75% of the lens.

In some embodiments, the sleep mask may include a connector adapted to connect the at least one LED to the fiber optic cable.

In another embodiment, the sleep mask may include a power source attached to the housing and electrically connected to provide power to the at least one LED. The power source may include a battery. The sleep mask may include a compartment for retaining the battery within the housing.

In some embodiments, the sleep mask may include control circuitry for controlling power from the battery to the at least one LED. The control circuitry may be included to allow a user to control various features of the sleep mask including, light transmissivity of the lens, light intensity and/or color of the at least one LED, or audio output from at least one speaker.

The sleep mask may further include at least one speaker attached to the housing for emitting sound to a user. The at least one speaker may be adjustably attached to the housing for slidable movement.

In other embodiments, the sleep mask may include a charging port for charging the power source. In another embodiment, the charging port may directly provide power to the sleep mask.

In some embodiments, the sleep mask may include a tracking device which may be arranged within a geolocation pocket attached to the housing.

The housing may include an outer portion and an inner portion. In some embodiments, the outer portion may include an outer layer, a middle layer, and an inner layer. In other embodiments, the inner portion may include an inner fabric layer, a foam layer, and an outer fabric layer. In another embodiment, the outer portion and the inner portion may be removably attached. The inner portion may be washable. The inner portion may include a cooling material.

In some embodiments, the sleep mask may include a biometric sensor attached to the housing. The biometric sensor may be configured to monitor at least one of a heart rate, a body temperature, sleep patterns, activity levels, or a respiratory rate of a user.

In accordance with another embodiment of the present invention, the silicone member may have a single channel therein for retaining a fiber optic cable such that light from at least one the associated LEDs is transmitted to a user.

A sleep mask system in accordance with an embodiment of the present invention includes a sleep mask in accordance with the present invention having selectively controlled features through the use of an application. The application may be adapted to be used from a remote device. In another aspect, the application may be adapted to control at least one of a light intensity of the at least one LED, a color of the at least one LED, a light transmission rate of the lens, or an audio output from at least one speaker attached to the housing.

### BRIEF DESCRIPTION OF THE DRAWINGS

The drawings are briefly described as follows.

FIG. 1 is a front perspective view of a sleep mask in accordance with an embodiment of the present invention.

FIG. 2A is a partially exploded front perspective view of the sleep mask in accordance with an embodiment of the present invention.

FIG. 2B is a partially exploded rear perspective view of the sleep mask in accordance with an embodiment of the present invention.

FIG. 3A is a rear perspective view of an outer portion of the sleep mask when in an opened state in accordance with an embodiment of the present invention.

FIG. 3B is a front perspective view of an inner portion of the sleep mask when in an opened state in accordance with an embodiment of the present invention.

FIG. 4 is a partial cross-sectional view of an outer portion and an inner portion of the sleep mask taken along line A-A of FIG. 1 when in an opened state in accordance with an embodiment of the present invention.

FIG. 5 is an isolated rear perspective view of a silicone member of the sleep mask in accordance with an embodiment of the present invention.

FIG. 5A is a partial cross-sectional view of a silicone member of the sleep mask taken along line B-B of FIG. 5 in accordance with an embodiment of the present invention.

FIG. 5B is a partial cross-sectional view of a silicone member of the sleep mask taken along line C-C of FIG. 5 in accordance with an embodiment of the present invention.

FIG. 6 is an isolated rear perspective view of a silicone member and a lens of the sleep mask in accordance with an embodiment of the present invention.

FIG. 6A is a partial cross-sectional view of a silicone member, a lens, and a fiber optic cable of the sleep mask taken along line D-D of FIG. 6 in accordance with an embodiment of the present invention.

FIG. 6B is a partial cross-sectional view of a silicone member, a lens, and a fiber optic cable of the sleep mask taken along line E-E of FIG. 6 in accordance with an embodiment of the present invention.

FIG. 7 is a partially disassembled view of the sleep mask showing the electrical components in accordance with an embodiment of the present invention.

FIGS. 8A to 8D are depictions of an application of a sleep mask system in accordance with the present invention.

The present invention now will be described more fully hereinafter with reference to the accompanying drawings, which show example embodiments. However, the present invention is not limited to the example embodiments set forth herein. Those skilled in the art will appreciate that various modifications can be made in the present disclosure without departing from the scope or spirit of the invention, which is set out in the claims.

### DETAILED DESCRIPTION

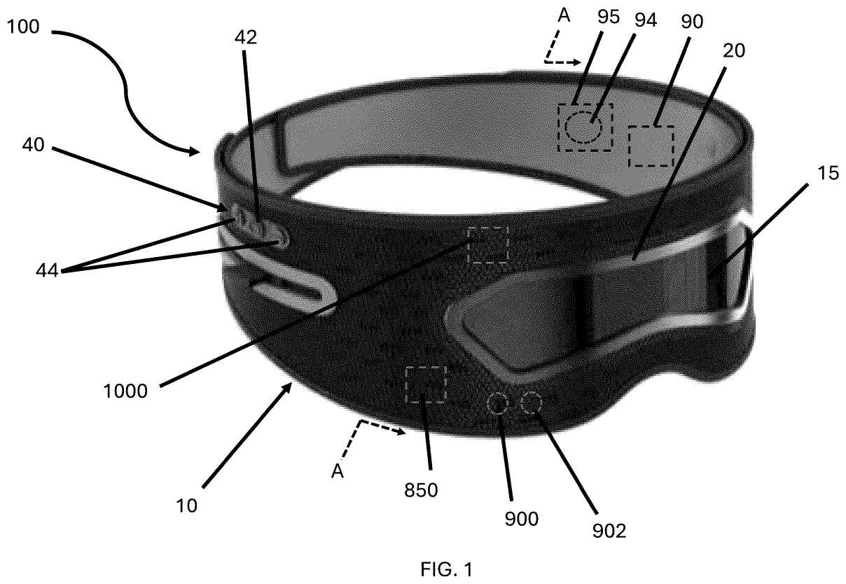

An embodiment of a sleep mask 100 of the present invention is shown in FIG. 1. The sleep mask 100 includes a housing 10 intended to be worn around a user's head. A silicone member 20 is attached to the housing 10 and retains a lens 15 in assembled position. A pair of LEDs, shown as a first LED 900 and a second LED 902, are schematically illustrated within the housing 10. The first and second LEDs 900 and 902 function to selectively provide light to a user to facilitate falling asleep and waking up as further discussed below. The sleep mask 100 includes a power source 850, which may be a battery or other power supply, is also schematically shown within the housing 10.

The silicone member 20 may be attached to the housing 10 by stitching, adhesive, clips, or other means. As shown in FIG. 1, the silicone member 20 may be arranged around the perimeter of the lens 15. In other embodiments, the silicone member may be attached to the housing to partially surround a perimeter of the lens. For example, the silicone member may surround at least 50%, at least 60%, at least 75%, at least 90%, or the entirety of the perimeter of the lens.

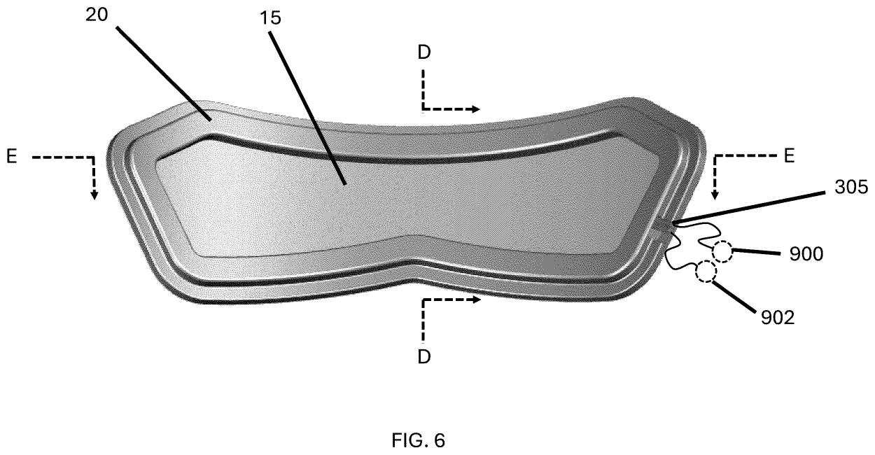

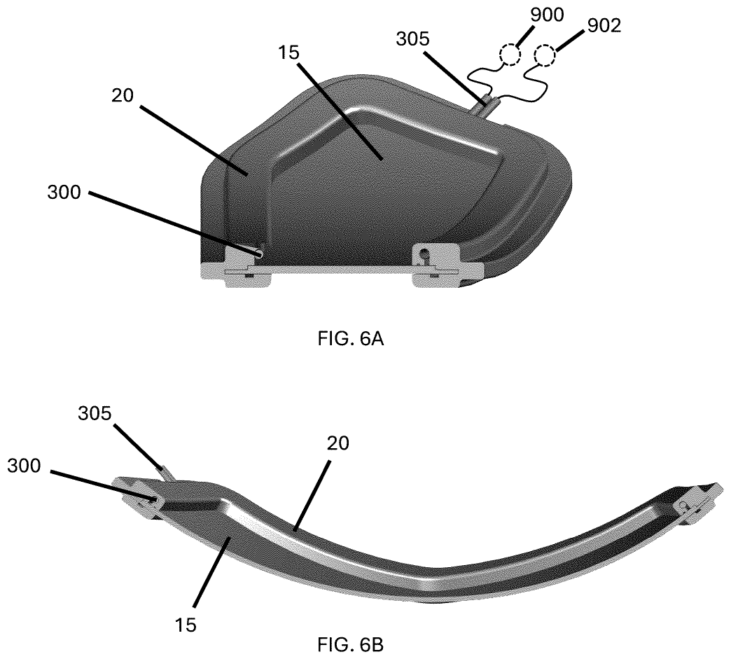

As also illustrated in FIG. 1, the silicone member 20 may retain a lens in assembled position through at least one channel included therein. Particularly, the silicone member 20 may include a first channel 105 and a second channel 115. In a preferred embodiment, as shown in FIGS. 6A and 6B described below, the lens 15 may be arranged within the first channel 105 and a fiber optic cable 300 may be arranged within the second channel 115. The lens 15 and the fiber optic cable 300 may be arranged within the first channel and the second channel, respectively, by friction, adhesive, stitching, clips or other means.

In an alternate embodiment, the silicone member 20 may not retain the lens 15 in assembled position. Rather, in such embodiments, the lens 15 may be attached to the housing 10 by other means such as adhesive, clips, stitching or the like.

The lens 15 may be made of polymer dispersed liquid crystal (PDLC) material. The lens 15 may be electrochromic and may thus be configured to undergo a change in opacity (i.e., light transmission rate) when exposed to an electric signal such as current or voltage from the power source 850. Particularly, when exposed to a selected current or voltage, the opacity of the lens 15 may vary from substantially translucent to substantially opaque. For example, the lens may have variable opacity (i.e., light transmission rate) ranging from 0% to 100%. In other embodiments, the lens may have a light transmission rate ranging from a low opacity range of between about 2%-25% to a high opacity range of between about 75%-98%. Within these ranges, the sleep mask may effectively facilitate a user falling asleep and waking up.

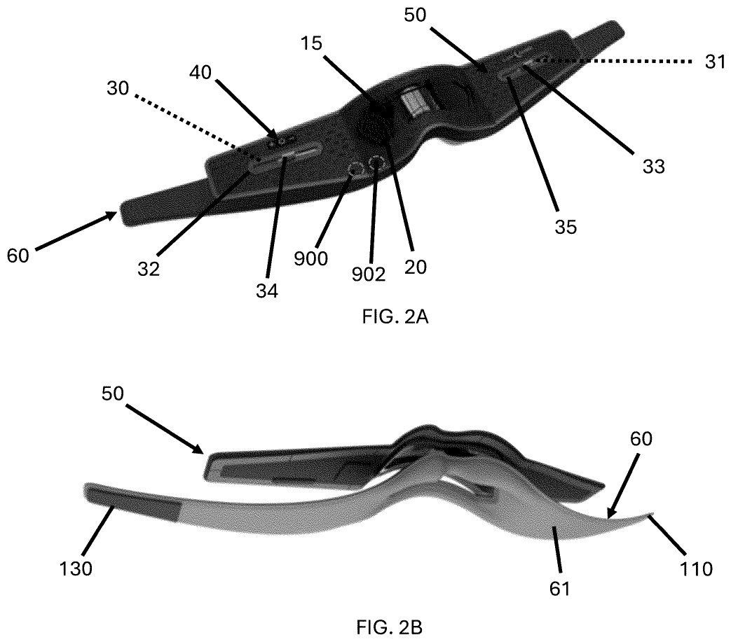

As schematically shown in FIGS. 1 and 2A, the sleep mask 100 includes a first LED 900 and a second LED 902. The first LED 900 and the second LED 902 may be secured to the housing 10. In some embodiments, the first LED 900 and the second LED 902 may be substantially adjacent to the silicone member 20. The first LED 900 and the second LED 902 may each be configured to transmit light having a separate color. The first LED 900 and the second LED 902 may also be configured to transmit light having a separate color of varying intensity (i.e., brightness) between off and full intensity.

In a preferred embodiment, the first LED 900 may be configured to transmit an orange light. The first LED may also be configured to transmit light having a wavelength ranging from about 590 nm to 620 nm or other wavelengths such as about 590 nm to 615 nm, about 590 nm to 610 nm, about 595 nm to 615 nm, about 595 nm to 610 nm, or about 595 nm to 605 nm. In one preferred embodiment, the first LED may be configured to transmit an orange light having a wavelength of about 600 nm. Within these ranges, the sleep mask may may effectively facilitate a user falling asleep.

The second LED 902 may be configured to transmit a blue light. The second LED 902 may also be configured to transmit light having a wavelength ranging from about 450 nm to 495 nm or other wavelengths such as about 450 nm to 490 nm, about 455 nm to 490 nm, about 460 nm to 490 nm, about 465 nm to 490 nm, about 470 nm to 490 nm, about 475 nm to 490 nm, about 480 nm to 490 nm, or about 480 nm to 485 nm. In a preferred embodiment, the second LED 902 may be configured to transmit a blue light having a wavelength of about 482 nm. Within these ranges, the sleep mask may effectively facilitate a user waking up.

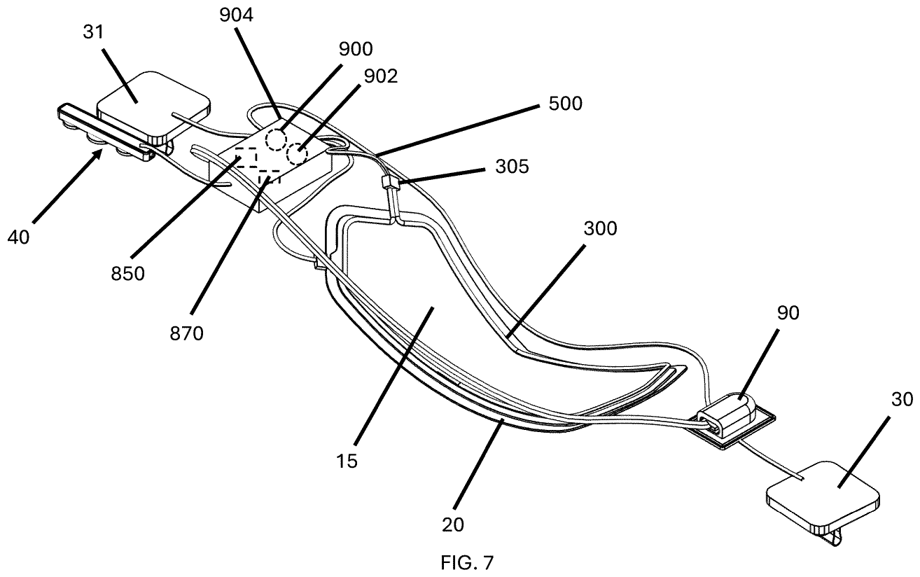

As schematically illustrated in FIG. 1, the sleep mask 100 may include a power source 850 attached to the housing 10. The power source 850 may include a battery. As shown in FIG. 7, the sleep mask may further include a compartment 904 for retaining the power source 850.

In some embodiments, the power source 850 may be electrically connected to provide power to various components of the sleep mask. Particularly, the power source 850 may be electrically connected to provide power to at least one of the first LED 900, the second LED 902, or the lens 15. In a preferred embodiment, the power source may also be electrically connected to provide power to at least one of a first speaker 30 or a second speaker 31 as further described below.

The sleep mask 100 is shown in FIG. 1 as having a control panel 40 attached to the housing 10. The control panel may include at least one of a power button 42 or volume control buttons 44. Particularly, the power button 42 may be configured to control electrical power from the power source 850 to at least one of the first LED 900, the second LED 902, or the lens 15. More particularly, the power button 42 may also be configured to control electrical power from the power source 850 to at least one of a first speaker 30 or a second speaker 31.

When pressed by a user, the power button 42 may transmit electrical signals from the power source 850 to at least one of the first LED 900, the second LED 902, the lens 15, the first speaker 30, or the second speaker 31. Further, when pressed by a user, the power button may also temporarily pause and/or restart a preset or saved scenario of the application 1100 of the sleep mask system as further described below. In other embodiments, when held down by a user (e.g., depressed and held for at least three seconds), the power button 42 may turn-off power to the sleep mask such that no power is provided from the power source 850 to any component of the sleep mask. In such embodiments, the power button may allow a user to control the electrical components of the sleep mask without the need of the application 1100 described below.

Volume control buttons 44 may allow a user to control the volume of audio (i.e., increase or decrease) from at least one of the first speaker 30 and the second speaker 31 without the need of the application 1100 of the sleep mask system described below.

As also schematically shown in FIG. 1, the sleep mask 100 may further include a biometric sensor 1000 attached to the housing 10. The biometric sensor may be configured to monitor at least one of a heart rate, a body temperature, sleep patterns, activity levels, or a respiratory rate of a user. The data obtained by the biometric sensor may be viewed by a user on the application 1100 of the sleep mask system described below. Accordingly, the data obtained by the biometric sensor 1000 may be useful for monitoring a user's sleeping patterns. Further, the data obtained by the biometric sensor may assist a user in diagnosing potential conditions (e.g., anxiety, depression, stress, and heart disease).

As illustrated in FIG. 2A, the sleep mask 100 may provide a relaxing audio experience for a user. In this embodiment, the sleep mask 100 may include a first speaker 30 and a second speaker 31 attached to the housing 10 to emit audio to a user. The first speaker 30 and the second speaker 31 may each be adjustably attached to the housing for slidable movement. Particularly, the first speaker 30 may include a first speaker slider 35 and a first tab 34. The first speaker slider 35 may allow a user to independently position the first speaker to optimize comfort and auditory experience. The first tab 34 may allow a user to easily adjust the position of the first speaker along the first speaker slider. Similarly, the second speaker 31 may include a second speaker slider 33 and a second tab 35. The second speaker slider 33 may allow a user to independently position the second speaker to optimize comfort and auditory experience. The second tab 35 may allow a user to easily adjust the position of the second speaker along the second speaker slider.

In one preferred embodiment, the first speaker 30 and the second speaker 31 may each be independently controlled through the control panel 40 or the application 1100 of the sleep mask system. Accordingly, the first speaker and the second speaker may facilitate relaxation, falling asleep, or waking up.

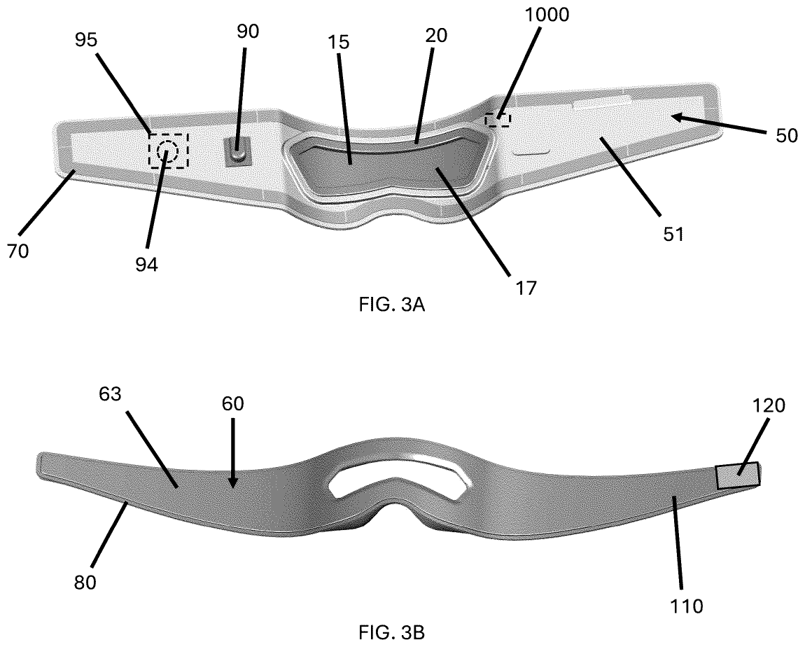

As shown in FIG. 3A, the sleep mask 100 may include a shatterproof plastic layer 17 laminated on one or both sides of the lens 15. The shatterproof plastic layer may reduce the risk of injury to a user if the lens breaks.

The sleep mask 100 may further include a tracking device 94 arranged in a geolocation pocket 95 within and attached to the housing 10. The tracking device 94 may be configured to detect the location of the sleep mask, which may be useful for auto-selection of a time-zone. The tracking device 94 may enable a user to pinpoint the location of the sleep mask 100 from a remote device. Particularly, the tracking device may enable a user to pinpoint the location of the sleep mask from the application 1100 of the sleep mask system further described below.

The sleep mask 100 may further include a charging port 90 attached to the housing 10. The charging port 90 may include any connector for providing power to the electronic components of the sleep mask 100, including a rechargeable battery of the power source 850 and other electronic components. It may include various connecting ports such as USB connector (for example, USB Type A, USB Type B, USB 3.0, USB Mini, USB Micro, USB Type C, or USB Micro B) to accommodate an appropriate charging cable. In a preferred embodiment, the charging port 90 may facilitate charging of the battery of power source 850. In other embodiments, the charging port 90 may be configured to directly provide power to other electronic components of the sleep mask. For example, in such embodiments, the charging port may be compatible with alternating current (AC) or direct current (DC) sources.

The charging port 90 may also be configured to wirelessly charge the power source 850 of the sleep mask 100 through various wireless protocols such as Bluetooth, WiFi, and other conventional and proprietary wireless protocols.

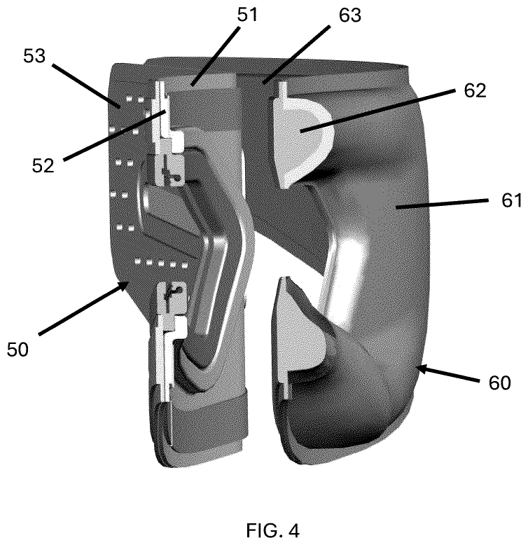

The housing 10 of the sleep mask 100 may be constructed out of materials that are flexible and comfortable to a user. As shown in FIGS. 3 and 4, the housing 10 may include an outer portion 50 and an inner portion 60 that are removably attached. FIG. 4 illustrates a partial cross-sectional view of the outer portion 50 and inner portion 60 of the sleep mask 100 taken along line A-A when shown in an open position.

The outer portion 50 of FIG. 4 includes an outer layer 51, a middle layer 52, and an inner layer 53. In one embodiment, the outer layer 51 may be made of a mesh fabric and the middle layer may be made of a foam material such as a polyurethane foam, a polyethylene foam, or a memory foam. Examples of acceptable fabric for the inner layer 53 include cotton, silk, wool, polyester, Rayon, and other natural or synthetic materials including woven and non-woven fabric.

The inner portion 60 may include an inner fabric layer 61, a foam layer 62, and an outer fabric layer 63. Acceptable fabrics and foam material may be the same or different form the materials used for the outer portion 50 discussed above. The inner portion 60 may be washable. The fabric should be selected for durability, comfort and washability.

In one embodiment, the inner portion 60 may include a cooling material such as a gel or other material to retain a cool temperature. The inner portion 60 may be adapted to at least partially apply a cool temperature to a user. Particularly, the inner portion 60 may be adapted to at least partially apply a cool temperature to the vagus nerve of a user to improve relaxation and sleep, as well as to reduce stress.

The electrical components of the sleep mask 100 include the first and second LEDs 900 and 902, the first and second speakers 30 and 31, the power source 850, and control circuitry. These electrical components may be attached to the outer portion 50 of the housing 10. In a preferred embodiment, the electrical components of the sleep mask 100 may be attached to the outer layer 51 of the housing.

As shown in FIGS. 3A and 3B, the outer portion 50 and the inner portion 60 may be removably attached to each other via a first attachment mechanism 70 and a second attachment mechanism 80. In a preferred embodiment, the first attachment mechanism may be located on the inner layer 51 of the outer portion 50. Further, the second attachment mechanism may be located on the outer fabric layer 63 of the inner portion 60. The first attachment mechanism and the second attachment mechanism may be removably attached to one another for ease of washing, cleaning, and/or repair.

The first attachment mechanism 70 and the second attachment mechanism 80 may be at least one of corresponding hook and loop fasteners (Velcro), snaps, clips, magnets, or other attachment means.

As also illustrated in FIGS. 3A and 3B, the first attachment mechanism 70 and second attachment mechanism 80 may surround a perimeter of the inner layer 51 and the outer fabric layer 63, respectively. In other embodiments, the first attachment mechanism and the second attachment mechanism may partially or fully surround a perimeter of the respective inner layer and outer fabric layer so that they are adequately secured to one another.

FIGS. 2B and 3B show that the inner portion 60 may further include an adjustable strap 110 configured to wrap around the head of a user. In a preferred embodiment, the adjustable strap 110 may further include a third attachment mechanism 120, attached to the outer fabric layer 63 of the inner portion 60, and a fourth attachment mechanism 130, attached to inner fabric layer 61 of the inner portion. The third attachment mechanism 120 and the fourth attachment mechanism 130 may be configured to secure the housing 10 around a user's head and face.

The third attachment mechanism and the fourth attachment mechanism may each be at least one of corresponding hook and loop fasteners, snaps, clips, or magnets. In a preferred embodiment, the third attachment mechanism and the fourth attachment mechanism may be at least one of corresponding hook and loop fasteners (Velcro), snaps, clips, magnets, or other attachment means.

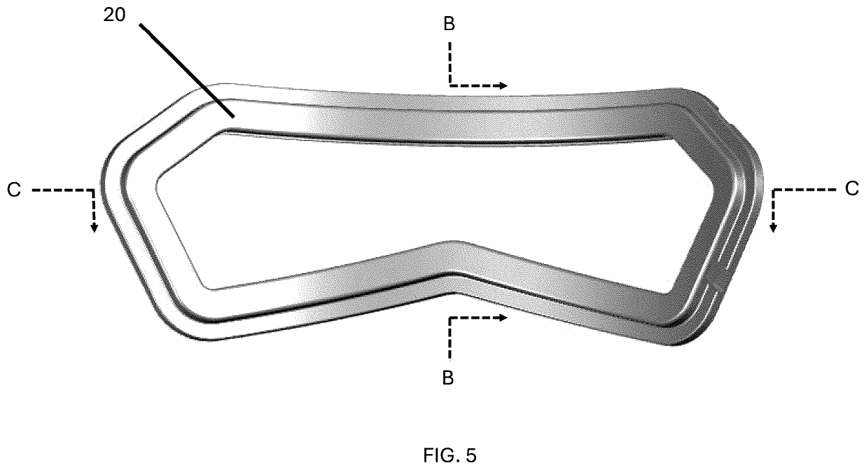

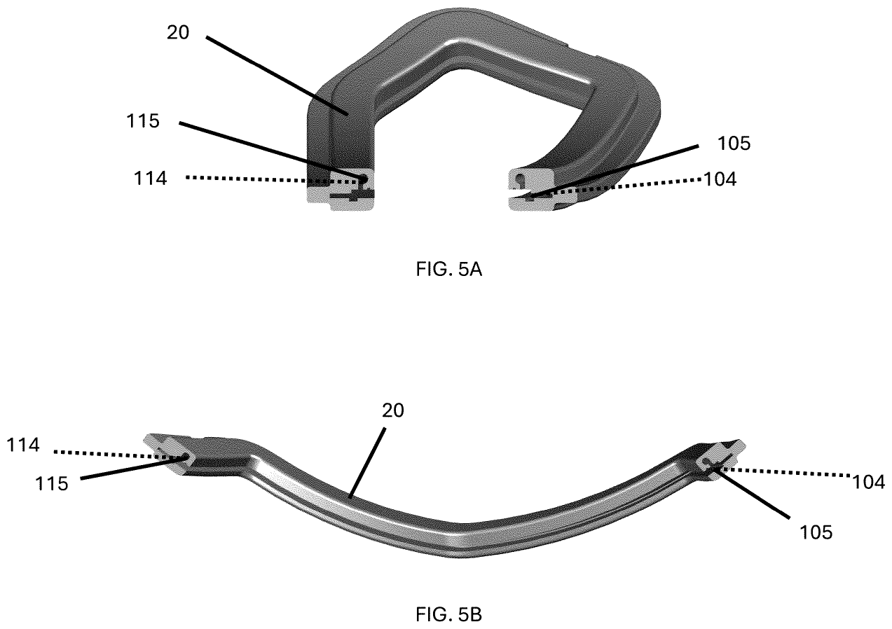

As shown in the partial cross-sectional views of FIG. 5A taken along line B-B of FIG. 5, and FIG. 5B taken along line C-C of FIG. 5, the silicone member 20 includes a first channel 105 and a second channel 115 therein. The first channel 105 may retain the lens 15 in assembled position and the second channel 115 may retain the fiber optic cable 300 in assembled position. It should be appreciated that the second channel 115 may extend along an entirety of a length of the silicone member 20 in which the fiber optic cable 300 is arranged.

In an aspect of the present invention, at least the outer surface of a portion (or all) of the first channel 105 may be coated with a substantially opaque composition. The first channel 105 may also be constructed out of a silicone material having a dark color, which is substantially opaque so that visible light waves are substantially or entirely blocked from transmission outside of the fiber optic cable 300. Preferably, in such embodiments, the first channel may be coated with a composition having a dark color such as black. In such embodiments, as depicted in FIG. 6B, the first channel 105 includes a solid region 104 which surrounds the first channel. In such embodiments, the solid region 104 may be substantially opaque. Preferably, the solid region 104 may have a black color.

In an embodiment of the present invention, the area 114 adjacent the second channel 115 of the silicone member 20 may be substantially translucent. Particularly, in some embodiments, the area surrounding the second channel 115 may be constructed out of a clear silicone material. Accordingly, in such embodiments, the second channel 115 may be adapted to uniformly transmit light from the fiber optic cable 300 around at least a portion of the periphery of the lens 15 to be seen by a user to effectively facilitate the user's experience of falling asleep and waking up.

As shown in the partial cross-sectional views of FIG. 6A taken along line D-D of FIG. 6, and FIG. 6B taken along line E-E of FIG. 6, the sleep mask 100 includes a fiber optic cable 300 and a connector 305 adapted to connect the first LED 900 and the second LED 902 to the fiber optic cable 300. The fiber optic cable 300 functions to facilitate efficient transmission of light from the first LED 900 and the second LED 902 around the periphery of lens 15 to create a scientifically and pleasing experience to a user. The structure of acceptable fiber optic cables is known in the art. For example, the fiber optic cable may be a side-emitting fiber optic cable and may include fiber optic strands.

The fiber optic cable 300 is shown in FIGS. 6A and 6B arranged within the second channel 115 of the silicone member 20. The fiber optic cable 300 is preferably arranged around a portion of, or the entire, perimeter of the lens 10. For example, the fiber optic cable 300 may be arranged around at least 25%, 50%, 60%, 70%, 80%, or 90% of the lens 10. In further embodiments, the fiber optic cable may be arranged around 50% to 100% of the perimeter of lens 15. Light from the first and second LEDs 900 and 902 may be uniformly transmitted to a user to facilitate falling asleep or waking up.

The electrical components of the sleep mask 100 are as shown in FIG. 7. The electrical components include control circuitry 870 within or attached to the housing 10. Particularly, the control circuitry will be arranged on a circuit board and electrically connected to the power source 850. In some embodiments, the control board may be configured to receive an electronic signal from the application 1100 described below. The electronic signal may be any wireless electronic signal. For example, the electronic signal may be a Bluetooth signal, a Wi-Fi signal, or may have any acceptable form of radio waves for communication. Further, in such embodiments, the control circuitry 870 may also include a microphone configured to detect ambient noise and noise cancellation circuitry such that the audio sound emitted from first and second speakers 30 and 31 is clear and pleasing to a user.

FIG. 7 also shows that the sleep mask 100 may include a further control circuit 500 attached to the housing 10. The control circuit may be configured to control power from the power source 850 to at least one of the first LED 900, the second LED 902, the lens 15, the first speaker 30, or the second speaker 31.

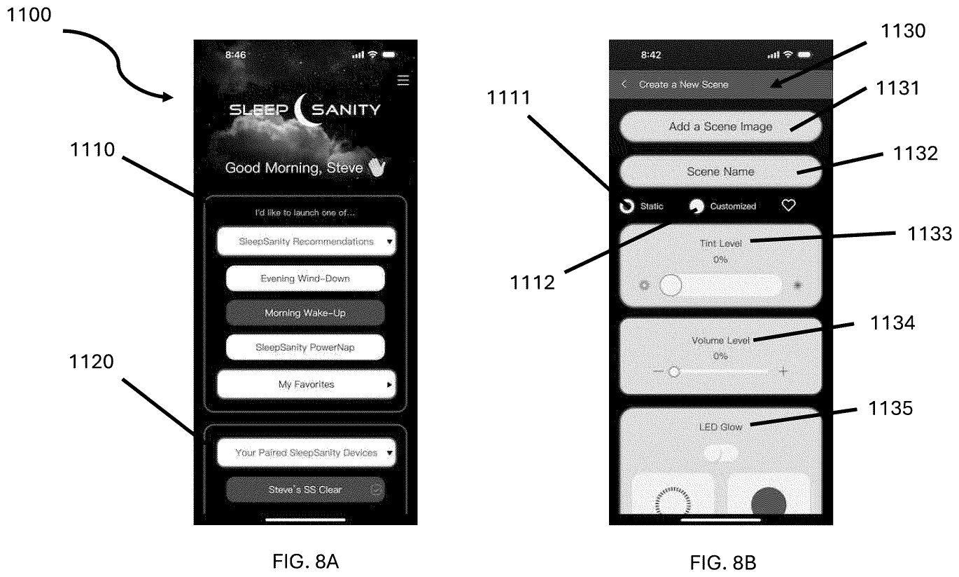

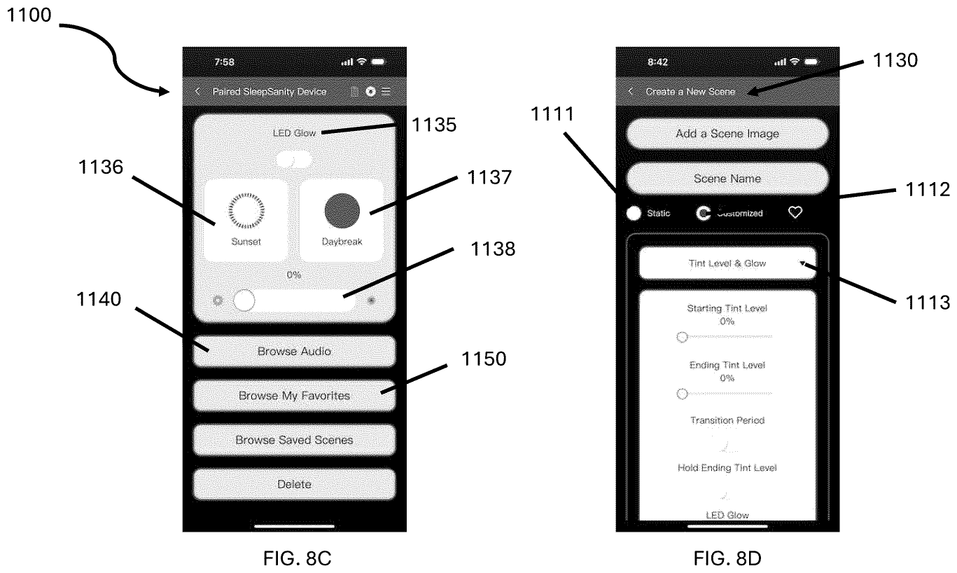

FIGS. 8A-8D illustrate features of an app for an embodiment of a sleep mask system 2000. The sleep mask system 2000 includes a sleep mask in accordance with the present disclosure and an application 1100. Sleep masks in accordance with the present disclosure have been described with reference to FIGS. 1-7, and thus share the detailed description provided above.

The sleep mask system 2000 may include an application 1100 adapted to be used from a remote device such as a smartphone, a smart watch, a smart TV, a laptop, a tablet, other computing device or the like.

As shown in FIGS. 8A-8D, the application 1100 may be configured to communicate with the control circuitry 870 of the sleep mask 100. Particularly, the control circuitry may be configured to receive a wireless signal from the application 1100. In such embodiments, the application 1100 may control activation and at least one of a light intensity of the first 900 or second 902 LEDs, a color of the first or second LEDs, a light transmission rate of the lens 10, an audio output from at least one of the first 30 or second 31 speakers, or electrical power from the power source 850 to at least one of the first or second LEDs, the lens, or the speakers.

The application 1100 may also include a scenario selector 1110. The scenario selector 1110 permits a user to select among preset scenarios or saved scenarios. For example, as depicted in FIG. 8A, the scenario selector permits a user to select among preset “Evening Wind-Down,” “Morning Wake-Up,” and “SLEEP SANITY PowerNap” scenarios. Additionally, the scenario selector may allow a user to select a customized saved scenario from a tab entitled “My Favorites. ” For ease of description, the term “preset scenarios” refers to pre-installed scenarios included in the application 1100. Particularly, “preset scenarios” may facilitate falling asleep, waking up, or relaxation according to norms of an average user. In contrast, “saved scenarios” may be scenarios preferred by a user and which have been customized to facilitate falling asleep, waking up, or relaxation.

As also illustrated in FIG. 8A, the application 1110 may include a mask selector 1120 to permit a user to pair one or more of the sleep masks 100 with the application.

FIGS. 8B-8D illustrate that the application may include a scenario creator 1130 adapted to enable a user to customize saved scenarios. The scenario creator may include at least one of a screen image selector 1131, a scenario name selector 1132, a light transmission rate selector 1133, a volume selector 1134, a LED glow selector 1135, a sunset glow selector 1136, a daybreak glow selector 1137, or a LED intensity selector 1138.

The screen image selector 1131 is shown in FIG. 8B and permits customization of a saved scenario with an image or thumbnail. In particular, the screen name selector 1132 permits a user to customize the name of a saved scenario. The light transmission rate selector 1133 permits customization of the light transmission rate of the lens 10. A light transmission rate selector may include an adjustable slider for allowing a user to control the light transmission rate (i.e., tint level) of the lens to range from 0% to 100%. The volume selector 1134 permits a user to customize the audio volume from at least one of first speaker 30 or the second speaker 31. The volume selector includes an adjustable slider for allowing a user to control the volume of at least one of the first speaker or the second speaker to range from 0% to 100%. The LED glow selector 1135 facilitates control over whether power from the power source 850 is provided to the first or the second LED.

FIG. 8C discloses a sunset glow selector 1136 to control whether power from the power source 850 is provided to the first LED. In a preferred embodiment, the sunset glow selector 1136 permits a user to control whether the first LED transmits an orange light, which will facilitate falling asleep. Similarly, the daybreak glow selector 1137 permits a user to control whether power from the power source is provided to the second LED, which in a preferred embodiment, transmits a blue light to facilitate waking up. In some embodiments, the LED intensity selector 1138 may allow a user to customize the light intensity from the first LED 900 or second LED 902. The LED intensity selector 1138 may include an adjustable slider for allowing a user to control the light intensity of the first or second LEDs.

FIG. 8C shows that the application 1110 may include an audio selector 1140 to allow a user to select among nature sounds, white noise, classical music, or any other audio to be emitted from at least the first speaker 30 or the second speaker 31. Particularly, the audio selector 1140 may allow a user to facilitate falling asleep, waking up, or relaxation, independently, or in tandem with a preset or saved scenario.

In some embodiments, the scenario creator 1130 may include a static scenario selector 1111, which permits a user to create a saved scenario without adjusting a light intensity of the first or second LEDs, a color of the first or the second LEDs, a light transmission rate of the lens 10, or an audio output from at least one of the first speaker 30 or the second speaker 31.

FIG. 8D discloses a further preferred embodiment where the scenario creator 1130 includes a customized scenario selector 1112 that permits a user to create a saved scenario that changes at least one of a light intensity of the first LED 900 or the second LED 902, a color of the first LED or the second LED, a light transmission rate of the lens 10, or an audio output from at least one of the first speaker 30 or the second speaker 31 over time.

As shown in FIG. 8D, once the customized scenario selector 1112 is selected, the scenario creator 1130 may include a customized drop down tab 1113 to permit a user to control at least one of a wake-up time, a wind-down time, an intensity of light from the first LED 900 or the second LED 902, a color of the first or second LED, a duration of the light from the first or second LED over time, a change in light transmission rate of the lens 10 over time, a holding time of a specific light transmission rate, an audio outputted from at least one of the first speaker 30 or the second speaker 31, or a change in volume of at least one of the first speaker or the second speaker. The customizable parameters may each include an adjustable slider for allowing a user to variably adjust each parameter to be in accord with a user's particular needs.

It is understood that when an element is referred hereinabove as being on or attached to another element, it can be directly or indirectly on the other element or intervening elements may be present therebetween.

Features illustrated or described as part of one embodiment can be used with another embodiment and such variations come within the scope of the appended claims and their equivalents. Implementations may also include one or a combination of any two or more of the aforementioned features or embodiments. As the invention in accordance with the present disclosure has been described in connection with what is presently considered to be practical embodiments, it is to be understood that the disclosure is not to be limited to the disclosed embodiments, but on the contrary, is intended to cover various modifications and equivalent arrangements included within the scope of the claims.

1. A sleep mask comprising:
(a) a housing adapted to be worn by a user;
(b) at least one LED attached to the housing;
(c) a power source attached to the housing and electrically connected to provide power to the at least one LED;
(d) a silicone member attached to the housing, the silicone member having at least a first channel and a second channel;
(e) a lens arranged within the first channel, the lens having adjustable opacity for transmitting or blocking light waves;
(f) a fiber optic cable arranged within the second channel for transmitting light from the at least one LED around at least a portion of the lens; and
(g) a connector adapted to connect the at least one LED to the fiber optic cable.

2. The sleep mask of claim 1 wherein the at least one LED comprises a first LED and a second LED, each configured to transmit light having a separate color.

3. The sleep mask of claim 2 wherein the first LED is configured to transmit an orange light, and the second LED is configured to transmit a blue light.

4. The sleep mask of claim 2 wherein the first LED is configured to transmit light having a wavelength ranging from about 590 nm to 610 nm and the second LED is configured to transmit light having a wavelength ranging from about 470 nm to 490 nm.

5. The sleep mask of claim 1 wherein the power source comprises a battery and a compartment for retaining the battery within the housing.

6. The sleep mask of claim 5 further comprising control circuitry for controlling power from the battery to the at least one LED.

7. The sleep mask of claim 1 wherein the lens comprises polymer dispersed liquid crystal (PDLC).

8. The sleep mask of claim 1 wherein the fiber optic cable is arranged around at least 50% of the lens.

9. The sleep mask of claim 1 wherein the fiber optic cable is arranged around at least 75% of the lens.

10. The sleep mask of claim 1 wherein the first channel is substantially opaque, and the second channel is substantially translucent.

11. The sleep mask of claim 1 further comprising at least one speaker attached to the housing for emitting audio to a user.

12. The sleep mask of claim 11 wherein the at least one speaker is adjustably attached to the housing for slidable movement.

13. The sleep mask of claim 1 further comprising a charging port for charging the power source.

14. The sleep mask of claim 1 further comprising a tracking device and a geolocation pocket attached to the housing, the tracking device being arranged within the geolocation pocket.

15. The sleep mask of claim 1 wherein the housing comprises an outer portion and an inner portion, wherein the outer and inner portions are removably attached.

16. The sleep mask of claim 15 wherein the inner portion is washable.

17. The sleep mask of claim 1 further comprising a biometric sensor attached to the housing and configured to monitor at least one of a heart rate, a body temperature, sleep patterns, activity levels, or a respiratory rate of a user.

18. A sleep mask comprising:
(a) a housing adapted to be worn by a user;
(b) at least one LED attached to the housing;
(c) a power source attached to the housing and electrically connected to provide power to the at least one LED;
(d) a silicone member attached to the housing, the silicone member having a channel;
(e) a lens attached to the housing, the lens having adjustable opacity for transmitting or blocking light waves;
(f) a fiber optic cable arranged within the channel for transmitting light from the at least one LED around at least a portion of the lens; and
(g) a connector adapted to connect the at least one LED to the fiber optic cable.

19. A sleep mask system, comprising:
(a) a sleep mask including, a housing adapted to be worn by a user, at least one LED attached to the housing, a power source attached to the housing and electrically connected to provide power to the at least one LED, a silicone member attached to the housing, the silicone member having at least a first channel and a second channel, a lens arranged within the first channel wherein the lens has adjustable opacity for transmitting or blocking light waves, a fiber optic cable arranged within the second channel for transmitting light from the at least one LED around at least a portion of the lens, and a connector adapted to connect the at least one LED to the fiber optic cable; and
(b) an application adapted to be used from a remote device to control at least one of a light intensity of the at least one LED, a color of the at least one LED, a light transmission rate of the lens, or an audio output from at least one speaker attached to the housing.

20. The sleep mask system of claim 19 wherein the at least one LED comprises a first LED and a second LED, each configured to transmit light having a separate color, and wherein the first LED is configured to transmit an orange light, and the second LED is configured to transmit a blue light.
